(19) United States
(12) Patent Application Publication
(10) Pub. No.: US 2020/0009248 A1
(43) Pub. Date: Jan. 9, 2020

(54) TARGETING TGR5 TO TREAT DISEASE

(71) Applicant: Mayo Foundation for Medical Education and Research, Rochester, MN (US)

(72) Inventors: Tetyana V. Masyuk, Rochester, MN (US); Nicholas F. LaRusso, Rochester, MN (US); Anatoliy Masyuk, Rochester, MN (US)

(73) Assignee: Mayo Foundation for Medical Education and Research, Rochester, MN (US)

(21) Appl. No.: 16/521,055

(22) Filed: Jul. 24, 2019

Related U.S. Application Data
(63) Continuation of application No. 15/516,499, filed on Apr. 3, 2017, now abandoned.
(60) Provisional application No. 62/059,530, filed on Oct. 3, 2014.

(51) Int. Cl. A61K 39/395; A61P 1/16; A61K 38/16; C07K 16/28

(52) U.S. Cl. CPC A61K 39/395; A61P 1/16; C07K 2317/76; C07K 16/28; A61K 38/16

(57) ABSTRACT

Materials and methods for treating cholangiopathies by targeting TGR5, including materials and methods for treating cholangiopathies such as polycystic liver disease, are provided herein.

### CROSS REFERENCE TO RELATED APPLICATIONS

This application is a continuation of U.S. application Ser. No. 15/516,499, filed Apr. 3, 2017, now abandoned, which is a National Stage Application under 35 U.S.C. § 371 that claims the benefit of Application Serial No. PCT/US2015/053222, filed Sep. 30, 2015, which claims the benefit of U.S. Provisional Ser. No. 62/059,530, filed Oct. 3, 2014. The disclosures of the prior applications are considered part of (and are incorporated by reference in) the disclosure of this application.

### STATEMENT AS TO FEDERALLY SPONSORED RESEARCH

This invention was made with government support under DK024031 awarded by the National Institutes of Health. The government has certain rights in the invention.

### TECHNICAL FIELD

This document relates to materials and methods for treating cholangiopathies by targeting TGR5, and more particularly to materials and methods for targeting TGR5 to treat cholangiopathies such as polycystic liver disease.

### BACKGROUND

The intrahepatic bile ducts make up a complex three-dimensional network of conduits within the liver, lined by specialized epithelial cells called cholangiocytes. A major physiological function of cholangiocytes is bile formation. From a pathological point of view, cholangiocytes represent the primary cell target of a diverse group of genetic and acquired biliary disorders, collectively called “cholangiopathies.” Cholangiopathies include primary biliary cirrhosis (PBC), graft vs. host disease (GVHD), post-transplant hepatic artery stenosis, chronic liver transplant rejection, cholangiocarcinoma, and genetically transmitted or developmental diseases such as cystic fibrosis, Alagille's syndrome, biliary atresia, and fibropolycystic diseases. Polycystic liver (PLD) disease and polycystic kidney disease (PKD) are genetic pathological conditions characterized by the formation of fluid-filled cysts in the liver and kidney, respectively. Current pharmacological management of these diseases shows short-term and/or modest beneficial effects.

### SUMMARY

This document is based at least in part on the elucidation of underlying molecular mechanisms involved in PLD/PKD pathogenesis, and the identification of TGR5 as a target for therapy of these conditions and other cholangiopathies. Thus, this document provides materials and methods for treating PLD, PKD, and other cholangiopathies by targeting TGR5.

In one aspect, this document features a method for treating polycystic liver disease in a subject. The method can include administering to the subject a TGR5 antagonist in an amount effective to reduce at least one symptom of the polycystic liver disease. The subject can be a human. The antagonist can be an antibody targeted to TGR5, or can be a small molecule.

In another aspect, this document features a method for reducing, inhibiting, or preventing cyst formation in the liver or kidney of a subject. The method can include administering to the subject a TGR5 antagonist in an amount effective to reduce the size or number of cysts in the liver or kidney of the subject. The subject can be diagnosed with PLD. The subject can be a human. The antagonist can be an antibody targeted to TGR5, or can be a small molecule.

This document also features the use of a TGR5 antagonist for treating PLD, where the TGR5 antagonist is for administration in an amount effective to reduce at least one symptom of the polycystic liver disease. The antagonist can be an antibody targeted to TGR5, or can be a small molecule.

In another aspect, this document features the use of a TGR5 antagonist for reducing cyst formation in the liver or kidney of a subject, where the TGR5 antagonist is for administration in an amount effective to reduce the size or number of cysts in the liver or kidney of the subject. The subject can be diagnosed with PLD. The subject can be a human. The antagonist can be an antibody targeted to TGR5, or can be a small molecule.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show that TGR5 message and protein levels are increased in cystic cholangiocytes. FIG. 1A is a graph plotting copy numbers of TGR5 transcript assessed by genome-wide sequencing, while FIG. 1B is a picture of a western blot showing levels of TGR5 protein in rat and human cystic cholangiocytes. FIG. 1C is a series of immunofluorescent images showing TGR5 overexpression in cholangiocytes lining liver cysts of wild type rodents, healthy humans, and rodents or patients with PLD, as indicated. TGR5 is stained in green, and nuclei are in blue. n=5 for each data set. *p<0.05 compared to respective controls.

FIG. 2A is a series of immunofluorescent images showing TGR5 expression in cilia of control and cystic cholangiocytes. TGR5 is localized to cholangiocyte cilia of wild type rat, mice and healthy human beings. In contrast, no TGR5 immunoreactivity is observed in cilia of cystic cholangiocytes. Magnification, ×100. TGR5 is in green, cilia are stained with acetylated α-tubulin (red), and nuclei are in blue. FIG. 2B is a pair of immuno-gold transmission electron microscopy (IG-TEM) images (top) showing expression of TGR5 in cilia of control but not PCK rats, and a graph (bottom) plotting the number of IG particles per cilia for a quantitative analysis. FIG. 2C is a pair of IG-TEM images (top) showing that the number of TGR5-positive IG particles is increased at the apical membrane of cystic cholangiocytes compared to control, and a graph (bottom) plotting the number of IG particles per cholangiocyte. Scale, 500 nm. L=lumen of bile duct (control rat) or cyst (PCK rat). C=control. n=3 for each data set. *, p<0.05 compared to respective controls.

FIGS. 3A-3E show that TGR5 activation differentially affects cAMP levels and cell proliferation in ciliated control vs. cystic cholangiocytes. FIG. 3A is a series of graphs plotting cAMP levels in control and cystic human and rat cholangiocytes treated with TGR5 agonists, as indicated. FIG. 3B is a series of graphs plotting cell proliferation in control and cystic human and rat cholangiocytes treated with TGR5 agonists, as indicated. In contrast, cAMP production and cell proliferation is increased in response to TGR5 activation in cystic cholangiocytes. FIG. 3C is a picture of representative western blots showing levels TGR5 protein expression in rat (PCK) and human (ADPKD) cystic cholangiocytes stably transfected with TGR5-shRNA or control shRNA. n=3 for each cell line. FIG. 3D is a pair of graphs plotting cAMP levels in rat (PCK) and human (ADPKD) cystic cholangiocytes in which TGR5 was depleted by shRNA. FIG. 3E is a pair of graphs plotting proliferation of rat (PCK) and human (ADPKD) cystic cholangiocytes in which TGR5 was depleted by shRNA. n=8 for each data set. Epidermal growth factor (EGF) was used as a positive control. *, p<0.05 compared to respective controls.

FIG. 4 is a series of graphs plotting cAMP levels in non-ciliated control and cystic rat and human cholangiocytes treated with TGR5 agonists as indicated. TLCA, taurolithocholic acid; OA, oleanolic acid; C1 and C2, two synthetic compounds. n=8 for each data set. *p<0.05 compared to respective controls.

FIG. 5 is a pair of graphs plotting levels of cAMP in cultured rat (top) and human (bottom) cholangiocytes in response to forskolin (FSK, 10⁻⁶ M) independently of the presence or absence of cilia. n=5 for each data set. *p<0.05 compared to respective controls.

FIG. 6 is a series of graphs plotting proliferation of control and cystic cholangiocytes in the absence of cilia after treatment with the indicated TGR5 agonists. EGF was used as a positive control. n=8 for each data set. *p<0.05 compared to respective controls.

FIGS. 7A and 7B show that TGR5 agonists increased growth of cystic structures in 3-D cultures. FIG. 7A is a series of representative images of PCK cystic bile ducts (left) and a scatter plot (right) demonstrating accelerated cyst expansion in the presence of TLCA and OA compared to untreated bile ducts. n=30 for each data set. FIG. 7B is a series of representative images (top) and a scatter plot (bottom) showing of cysts formed in 3-D culture by ADPKD cholangiocytes treated with TGR5 agonists as indicated, and by untreated controls, both with and without depletion of TGR5 by shRNA. TGR5 depletion in ADPKD cholangiocytes abolished the effects of TLCA and OA. Scale bar, 250 mm. n=20 for each data set.

FIGS. 8A and 8B show that oleanolic acid increases hepato-renal cystogenesis in PKC rats. FIG. 8A contains a pair of representative images of picrosirius red stained liver (top) from un-treated (control) and OA-treated PCK rats, as well as a trio of graphs plotting liver weight as a percentage of total body weight (left graph), and plotting cystic (center graph) and fibrotic (right graph) areas of individual liver lobes (three liver lobes from each rat) as a percentage of total liver parenchyma. FIG. 8B contains a pair of representative images of picrosirius red stained kidney sections (top) from un-treated (control) and OA-treated PCK rats, as well as a trio of graphs plotting kidney weight as a percentage of total body weight (left graph), and plotting cystic (center graph) and fibrotic (right graph) areas of individual kidneys (two kidneys from each rat) as a percentage of total kidney parenchyma. *, p<0.05.

FIG. 9 is a series of confocal images of liver sections immunostained with TGR5 antibody (green), demonstrating that TGR5 is increased in Pkhd1^{del2del2} mice (compared to wild type and TGR5^{-/-} mice), while being reduced in double mutant TGR5^{-/-}:Pkhd1^{del2/del2} mice as compared to Pkhd1^{del2/del2} counterparts. Insets show asterisked areas without nuclear staining. n=8 for each data set. Magnification, ×100. *p<0.05.

FIG. 10 shows that hepatic cystogenesis is reduced in double mutant TGR5^{-/-}:Pkhd1^{del2/del2} mice. The top panels are a series of images showing livers stained with picrosirius red; asterisks in the upper panels indicate the areas shown in the middle panels (magnification, ×4). Cysts are absent in wild type and TGR5^{-/-} rodents but present in Pkhd1^{del2/del2} mice. TGR5^{-/-}:Pkhd1^{del2/del2} double mutants have reduced hepatic cystogenesis. The bottom panel contains a trio of graphs plotting liver weight (left graph) as a percentage of total body weight, and hepatic cystic and fibrotic areas (middle and right graphs, respectively) as a percentage of total liver parenchyma. n=6 mice for each data set. *p<0.05.

FIGS. 11A-11C show that Gαᵢ and Gαₛ proteins are differentially expressed in cystic cholangiocytes. FIG. 11A is a picture of representative westerns blots indicating levels of Gαᵢ, Gαₛ, and actin control in control and cystic cholangiocytes treated with or without OA. Quantitative data are presented in FIG. 11B. Increased expression of Gαₛ proteins is observed in cystic cholangiocytes compared to Gαᵢ proteins (a*). In comparison with control cholangiocytes, levels of Gαᵢ proteins are decreased (b*), while levels of Gαₛ are increased (c*) in cystic cholangiocytes. OA has no effects on expression of Gαᵢ or Gαₛ proteins. FIG. 11C is a series of immunofluorescent images showing elevated levels of Gαₛ proteins in vivo in PCK rats. OA does not affect the expression of Gα proteins in PCK rats, but increases TGR5-Gαₛ protein coupling. n=3 for each data set. *, p<0.05 compared to respective controls. Magnification, ×63. TGR5 is stained in red, Gαᵢ and Gαₛ proteins are in green, and nuclei are counterstained with DAPI.

### DETAILED DESCRIPTION

Cholangiopathies include PBC, GVHD, PLD, post-transplant hepatic artery stenosis, chronic liver transplant rejection, cholangiocarcinoma, cystic fibrosis, Alagille's syndrome, biliary atresia, and fibropolycystic diseases. PLD is a genetic pathological disorder characterized by the formation of multiple cysts derived from cholangiocytes. PLD typically co-exists with autosomal dominant (AD-) or autosomal recessive (AR-) PDK. PKD and PLD belong to a group of diseases collectively known as ciliopathies—genetic disorders associated with structurally and functionally defective cilia in renal and hepatic epithelia due to aberrant expression of disease-related and ciliary-associated proteins (Wills et al., Trends Mol Med 2014, 50:260-270; Masyuk et al., Curr Opin Gastroenterol 2009, 25:265-271; and Tones and Harris, J Am Soc Nephrol: JASN 2014, 25:18-32). In particular, ADPKD is caused by mutations in the PKD1 and PKD2 genes, while mutations in PKHD1 gene are responsible for renal and hepatic cystogenesis in ARPKD. Isolated autosomal dominant PLD (ADPLD) is a rare condition caused by mutations in either the SEC63 gene or the PRKCSH gene (Wills et al., supra; Fedeles et al., Trends Mol Med 2014, 20:251-60; and Masyuk et al., Curr Opin Gastroenterol 2009, 25:265-271).

Beside ciliary abnormalities, disturbances in multiple intracellular mechanisms can contribute to hepatic cystogenesis, including increased cell proliferation, dysregulated cell cycle, enhanced fluid secretion, decreased intracellular calcium levels, and global changes in mRNA, microRNA (miRNA), and protein expression. These cellular mechanisms of cyst growth are regulated by the intracellular signaling messenger, cAMP, levels of which are markedly increased in cystic cholangiocytes (Masyuk et al., Gastroenterol 2003, 125:1303-1310). Several drugs (tolvaptan and somatostatin analogs) intended to lower cAMP have been tested in clinical trials of PLD and PKD, but they only showed modest effects on cyst growth.

TGR5 (GPBAR-1, M-Bar or GPR131) is a transmembrane G protein-coupled bile acid receptor linked to cAMP signaling. TGR5 is expressed in a variety of human and rodent tissues, and has been shown to regulate bile and energy homeostasis, inflammation, immune responses, insulin secretion, gallbladder relaxation, and metabolic events (Schaap et al., Gastroenterol Hepatol 2014, 11:55-67; Pols, Biochem Soc Trans 2014, 42:244-249; and Duboc et al., Digestive and Liver Disease: Official Journal of the Italian Society of Gastroenterology and the Italian Association for the Study of the Liver 2014, 46:302-312). TGR5 is activated in response to different bile acids (e.g., lithocholic, chenodeoxycholic, deoxycholic, and cholic acids), xenobiotic ligands (e.g., oleanolic acid), and multiple semi-synthetic derivatives (Schaap et al., supra; Pols, supra; and Li et al., Biochem Pharmacol 2013, 86:1517-1524). Activation of TGR5 affects intracellular cAMP via coupling to Gαₛ or Gαᵢ proteins, subsequently triggering downstream signaling events (Jensen et al., J Biol Chem 2013, 288:22942-22960; and Masyuk et al., Am J Physiol. Gastrointestinal and Liver Physiol 2013, 304:G1013-1024).

In the liver, TGR5 is found in sinusoidal cells, Kupffer cells, gallbladder epithelia, and cholangiocytes, but not in hepatocytes (Schaap et al., supra; Duboc et al., supra; Li et al., supra; and Keitel and Haussinger, Curr Opin Gastroenterol 2013, 29:299-304). In control cholangiocytes, TGR5 is localized to cellular compartments including the primary cilia, apical plasma membrane, intracellular vesicles, and nuclear membrane (Masyuk et al., Am J Physiol. Gastrointestinal Liver Physiol 2013; 304:G1013-1024; and Keitel and Haussinger, supra). Effects of TGR5 activation on cAMP production and cell proliferation in control cholangiocytes are cilia-dependent. In the absence of cilia, up-regulated cAMP levels and increased cell proliferation are observed in response to TGR5 agonists, while opposite effects are seen in ciliated cholangiocytes.

This document therefore provides materials and methods for using a TGR5 antagonist to treat a subject diagnosed as having a cholangiopathy as described herein (e.g., PLD or PKD), as well as materials and methods for using a TGR5 antagonist to reduce, slow, or prevent the formation or growth of cysts in the liver or kidney of a subject. The subject can be, for example, a human patient. In some cases, the subject can be a research animal (e.g., a mouse, rat, rabbit, dog, pig, sheep, or monkey).

Suitable TGR5 antagonists for use in the methods provided herein include, for example, small molecules, nucleic acids targeted to a TGR5 sequence, and antibodies.

Examples of small molecules include, for example, those disclosed in U.S. Pat. No. 8,796,249, which is incorporated herein by reference in its entirety.

In some embodiments, a TGR5 antagonist can be an agent that reduces the level of mRNA that encodes a TGR5 polypeptide. For example, a TGR5 antagonist can be an agent that reduces transcription of nucleic acid encoding a TGR5 polypeptide, or promotes degradation of mRNA encoding a TGR5 polypeptide (e.g., by RNA interference (RNAi)), or inhibits posttranscriptional processing (e.g., splicing or nuclear export) of mRNA encoding a TGR5 polypeptide. Such an antagonist can inhibit protein synthesis from TGR5 mRNA (e.g., by RNA interference), or promote degradation of TGR5 protein, thereby reducing the level of TGR5 polypeptide in a subject. For example, a TGR5 antagonist can be a small interfering RNA (siRNA) molecule. siRNAs can be synthesized in vitro or made from a DNA vector in vivo. In some cases, an siRNA molecule can contain a backbone modification to increase its resistance to serum nucleases and increase its half-life in the circulation. Such modification can be made as described elsewhere (Chiu et al., RNA 2003, 9:1034-1048; and Czauderna et al., Nucleic Acids Res 2003, 31:2705-2716). In some cases, a small hairpin RNA (shRNA, which can be converted to an siRNA) can be used as a TGR5 antagonist.

The term “antibody” includes monoclonal antibodies, polyclonal antibodies, recombinant antibodies, humanized antibodies (Jones et al., Nature 1986, 321:522-525; Riechmann et al., Nature 1988, 332:323-329; and Presta, Curr Op Struct Biol 1992, 2:593-596), chimeric antibodies (Morrison et al. Proc Natl Acad Sci USA 1984, 81:6851-6855), multispecific antibodies (e.g., bispecific antibodies) formed from at least two antibodies, and antibody fragments. The term “antibody fragment” comprises any portion of the afore-mentioned antibodies, such as their antigen binding or variable regions. Examples of antibody fragments include Fab fragments, Fab′ fragments, F(ab′)2 fragments, Fv fragments, diabodies (Hollinger et al. Proc Natl Acad Sci USA 1993, 90:6444-6448), single chain antibody molecules (Plückthun in: The Pharmacology of Monoclonal Antibodies 113, Rosenburg and Moore, eds., Springer Verlag, N.Y. (1994), 269-315) and other fragments as long as they exhibit the desired capability of binding to B7-H1.

The term “antibody,” as used herein, also includes antibody-like molecules that contain engineered sub-domains of antibodies or naturally occurring antibody variants. These antibody-like molecules may be single-domain antibodies such as V_{H}-only or V_{L}-only domains derived either from natural sources such as camelids (Muyldermans et al. (2001) Rev Mol Biotechnol 74:277-302) or through in vitro display of libraries from humans, camelids or other species (Holt et al. (2003) Trends Biotechnol 21:484-90). In certain embodiments, the polypeptide structure of the antigen binding proteins can be based on antibodies, including, but not limited to, minibodies, synthetic antibodies (sometimes referred to as “antibody mimetics”), human antibodies, antibody fusions (sometimes referred to as “antibody conjugates”), and fragments thereof, respectively.

An “Fv fragment” is the minimum antibody fragment that contains a complete antigen-recognition and -binding site. This region consists of a dimer of one heavy chain variable domain and one light chain variable domain in tight, non-covalent association. It is in this configuration that the three CDR's of each variable domain interact to define an antigen-binding site on the surface of the V_{H}-V_{L} dimer. Collectively, the six CDR's confer antigen-binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (C_{H}1) of the heavy chain. The “Fab fragment” differs from the “Fab′ fragment” by the addition of a few residues at the carboxy terminus of the heavy chain C_{H}1 domain, including one or more cysteines from the antibody hinge region. The “F(ab′)2 fragment” originally is produced as a pair of “Fab′ fragments” which have hinge cysteines between them. Methods of preparing such antibody fragments, such as papain or pepsin digestion, are known to those skilled in the art.

An antibody can be of the IgA-, IgD-, IgE-, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type.

In some embodiments, antibodies can be fully human or humanized antibodies. Human antibodies can avoid certain problems associated with xenogeneic antibodies, such as antibodies that possess murine or rat variable and/or constant regions. First, because the effector portion is human, it can interact better with other parts of the human immune system, e.g., to destroy target cells more efficiently by complement-dependent cytotoxicity or antibody-dependent cellular cytotoxicity. Second, the human immune system should not recognize the antibody as foreign. Third, half-life in human circulation will be similar to naturally occurring human antibodies, allowing smaller and less frequent doses to be given. Methods for preparing human antibodies are known in the art.

In addition to human antibodies, “humanized” antibodies can have many advantages. Humanized antibodies generally are chimeric or mutant monoclonal antibodies from mouse, rat, hamster, rabbit or other species, bearing human constant and/or variable region domains or specific changes. Techniques for generating humanized antibodies are well known to those of skill in the art. For example, controlled rearrangement of antibody domains joined through protein disulfide bonds to form new, artificial protein molecules or “chimeric” antibodies can be utilized (Konieczny et al. Haematologia (Budap.) 1981, 14:95). Recombinant DNA technology can be used to construct gene fusions between DNA sequences encoding mouse antibody variable light and heavy chain domains and human antibody light and heavy chain constant domains (Morrison et al. Proc Natl Acad Sci USA 1984, 81:6851).

DNA sequences encoding antigen binding portions or complementarity determining regions (CDR's) of murine monoclonal antibodies can be grafted by molecular means into DNA sequences encoding frameworks of human antibody heavy and light chains (Jones et al. Nature 1986, 321:522; and Riechmann et al. Nature 1988, 332:323). Expressed recombinant products are called “reshaped” or humanized antibodies, and comprise the framework of a human antibody light or heavy chain and antigen recognition portions, CDR's, of a murine monoclonal antibody.

Other methods for designing heavy and light chains and for producing humanized antibodies are described in, for example, U.S. Pat. Nos. 5,530,101; 5,565,332; 5,585,089; 5,639,641; 5,693,761; 5,693,762; and 5,733,743. Additional methods for humanizing antibodies are described in U.S. Pat. Nos. 4,816,567; 4,935,496; 5,502,167; 5,558,864; 5,693,493; 5,698,417; 5,705,154; 5,750,078; and 5,770,403, for example. All of the above patents are incorporated herein by reference in their entirety.

One or more TGR5 antagonists can be incorporated into a composition for administration to a subject (e.g., a research animal or a human patient diagnosed as having a cholangiopathy). For example, a TGR5 antagonist can be administered to a subject under conditions wherein the progression of cyst formation is reduced in a therapeutic manner. Compositions containing one or more TGR5 antagonists can be given once or more daily, weekly, monthly, or even less often, or can be administered continuously for a period of time (e.g., hours, days, or weeks). In some cases, preparations can be designed to stabilize the TGR5 antagonist(s) and maintain effective activity in a mammal for several days.

The TGR5 antagonist(s) to be administered to a subject can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption. In some cases, a composition to be administered can contain one or more TGR5 antagonists in combination with a pharmaceutically acceptable carrier. Pharmaceutically acceptable carriers include, for example, pharmaceutically acceptable solvents, suspending agents, or any other pharmacologically inert vehicles for delivering compounds to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water, saline solution, binding agents (e.g., polyvinylpyrrolidone or hydroxypropyl methylcellulose); fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

A TGR5 antagonist or a composition containing a TGR5 antagonist can be used in methods for treating cholangiopathies (e.g., PLD, PKD, GVHD, post-transplant hepatic artery stenosis, chronic liver transplant rejection, cystic fibrosis, Alagille's syndrome, or biliary atresia). In some embodiments, for example, a method can include administering to a subject (e.g., a patient diagnosed as having a condition such as PLD or PKD) a TGR5 antagonist, or a composition containing a TGR5 antagonist, in an amount that is effective to reduce at least one symptom of the condition. In some embodiments, a method as provided herein can be used to reduce/inhibit/prevent cyst formation in the liver or kidney of a subject; such methods can include administering to a subject a TGR5 antagonist, or a composition containing a TGR5 antagonist, in an amount effective to reducing the size or number of cysts in the liver or kidney of the subject.

In the methods provided herein, a TGR5 antagonist or a composition containing a TGR5 antagonist can be administered by any of a number of methods, including oral, subcutaneous, intrathecal, intraventricular, intramuscular, intraperitoneal, or intravenous injection, or elution from implanted devices/structures.

The methods of treatment provided herein can be performed in a variety of manners, such that an effective amount of a TGR5 antagonist is delivered. In some embodiments, for example, a method of treatment can include administration of a low dose (e.g., 1 ng/kg/day to 10 mg/kg/day, such as 5 ng/kg/day, 10 ng/kg/day, 50 ng/kg/day, 100 ng/kg/day, 500 ng/kg/day, 1 μg/kg/day, 5 μg/kg/day, 10 μg/kg/day, 50 μg/kg/day, 100 μg/kg/day, 500 μg/kg/day, 1 mg/kg/day, 2.5 mg/kg/day, or 5 mg/kg/day) of a TGR5 antagonist for an extended length of time (e.g., one week or more, two weeks or more, or four weeks or more).

In some embodiments, the methods provided herein can include intermittent treatment with a TGR5 antagonist. Such approaches can include administration of a relatively high dose (e.g., 10 mg/kg/day to 1 g/kg/day, such as 25 mg/kg/day, 50 mg/kg/day, 100 mg/kg/day, 250 mg/kg/day, 500 mg/kg/day, or 750 mg/kg/day) of a TGR5 antagonist for a short period of time (e.g., 0.5 day, one day, two days, three days, four days, 5 days, 6 days, or 7 days), which may result in a substantial reduction in cyst formation or growth. Such methods also may include a period of “recovery” that can prevent deleterious/unwanted side effects secondary to chronic treatment with a TGR5 antagonist.

An effective amount of a TGR5 antagonist as provided herein can be any amount that reduces a symptom of the condition being treated, without significant toxicity. For example, the amount of TGR5 antagonist administered to a subject can be effective to reduce one or more symptoms of cholangiopathic disease in the subject. For example, the amount of TGR5 antagonist administered can be effective to reduce or prevent the formation or growth of cysts in the liver or kidney of a PLD or PKD patient by at least 5 percent (e.g., at least 10 percent, at least 25 percent, at least 50 percent, at least 75 percent, or at least 90 percent), as compared to the formation or growth of cysts in the liver or kidney of a control subject (e.g., a PLD or PKD patient not treated with the TGR5 antagonist). The formation or growth of cysts can be assessed based on, for example, the number of cysts in a specified area or volume of tissue, or the liver or kidney volume, which can be assessed by CT scan. In some embodiments, the amount of TGR5 antagonist administered can be effective to reduce the level of cAMP in liver or kidney cells of a PLD or PKD patient by at least 5 percent (e.g., at least 10 percent, at least 25 percent, at least 50 percent, at least 75 percent, or at least 90 percent), as compared to the level of cAMP in liver or kidney cells of a control subject (e.g., a PLD or PKD patient not treated with the TGR5 antagonist).

This document also provides for the use of a TGR5 antagonist for treating PLD and/or for reducing cyst formation in the liver or kidney of a subject (e.g., a human patient). In addition, this document provides for the use of a TGR5 antagonist in the manufacture of a medicament for treating PLD and/or for reducing cyst formation in the liver or kidney of a subject (e.g., a human patient). The TGR5 antagonist may be for administration in an effective amount as described above, such that it reduces at least one symptom of the PLD, and/or reduces the size or number of cysts in the liver or kidney of the subject.

The invention will be further described in the following examples, which do not limit the scope of the invention described in the claims.

### EXAMPLES

### Example 1—Materials and Methods

Cell Cultures, Reagents and Animals.

Control and cystic rat cholangiocytes (derived from wild type and PCK rats, respectively), and control and cystic human cholangiocytes (derived from healthy humans and ADPKD patients, respectively) were used. Cells were isolated and maintained as described elsewhere (Masyuk et al., Hepatol 2013, 58:409-421). TGR5 activation was achieved with: (i) taurolithocholic acid (TLCA; Sigma-Aldrich, St. Louis, Mo.); (ii) oleanolic acid (OA; Sigma); (iii) compound “1” (C1)-(2-(ethylamino)-6-(3-(4-(trifluoromethoxy)phenyl)propanoyl)-5,6,7,8-tetrahydropyrido[4,3-d]pyrimidine-4-carbox-amide); and (iv) compound “2” (C2)—3-(2-chlorophenyl)-N-(4-chlorophenyl)-N,5-dimethylisoxazole-4-carboxamide. C1 and C2 were synthesized in the Sanford-Burnham Medical Research Institute at >95% purity by HPLC following a procedure outlined elsewhere (Piotrowski et al., ACS Med Chem Lett 2013, 4:63-68; and Evans et al., J Med Chem 2009, 52:7962-7965). PCK and ADPKD cholangiocytes were stably transfected with TGR5 shRNAs or control shRNAs (Santa Cruz Biotechnology, Santa Cruz, Calif.). Rodents were maintained on a standard diet and water ad lib. After anesthesia with pentobarbital (50 mg/kg), liver and kidney were fixed and paraffin-embedded.

RNA Preparation and Sequencing.

RNA-sequencing was performed using cholangiocytes isolated from wild type and PCK rats, healthy human beings, and patients with ADPKD (n=3 for each condition). Total RNA was extracted with TRIZOL® (Invitrogen, Carlsbad, Calif.) followed by Agilent quality assessment (Agilent Technologies, Santa Clara, Calif.). RNA libraries were prepared according to the manufacturer's instructions for the TRUSEQ® RNA Sample Prep Kit v2 (Illumina, San Diego, Calif.). The liquid handling Eppendorf (Hamburg, GER) EPMOTION® 5075 robot was employed for TRUSEQ® library construction. AMPure bead clean up, mRNA isolation, end repair, and A-tailing reactions were completed on the 5075 robot. Reverse transcription and adaptor ligation steps were performed manually. Briefly, poly-A mRNA was purified from total RNA using oligo dT magnetic beads. The purified mRNA was fragmented at 95° C. for 8 minutes, eluted from the beads and primed for first strand cDNA synthesis. The RNA fragments were then copied into first strand cDNA using SuperScript III reverse transcriptase and random primers (Invitrogen). Next, second strand cDNA synthesis was performed using DNA polymerase I and RNase H. The double-stranded cDNA were purified using a single AMPure XP bead (Agencourt, Danvers, Mass.) clean-up step. The cDNA ends were repaired and phosphorylated using Klenow, T4 polymerase, and T4 polynucleotide kinase followed by a single AMPure XP bead clean-up. The blunt-ended cDNAs were modified to include a single 3′ adenylate (A) residue using Klenow exo- (3′ to 5′ exo minus). Paired-end DNA adaptors (Illumina) with a single “T” base overhang at the 3′ end were immediately ligated to the ‘A tailed’ cDNA population. Unique indexes, included in the standard TRUSEQ® Kits (12-Set A and 12-Set B) were incorporated at the adaptor ligation step for multiplex sample loading on the flow cells. The resulting constructs were purified by two consecutive AMPure XP bead clean-up steps. The adapter-modified DNA fragments were then enriched by 12 cycles of PCR using primers included in the Illumina Sample Prep Kit. The concentration and size distribution of the libraries were determined on an Agilent Bioanalyzer DNA 1000 chip (Santa Clara, Calif.). A final quantification, using Qubit fluorometry (Invitrogen), was done to confirm sample concentration. Libraries were loaded onto paired end flow cells at concentrations of 8-10 pM to generate cluster densities of 700,000/mm² following Illumina's standard protocol using the Illumina cBot and cBot Paired end cluster kit v3. The flow cells were sequenced as 51X2 paired end reads on an Illumina HiSeq 2000 using TRUSEQ® SBS sequencing kit v3 and HCS v2.0.12 data collection software. Base-calling was performed using Illumina's RTA version 1.17.21.3. Sequencing data were processed using the MAP-RSeq pipeline (Mayo Clinic Bioinformatics Core; Kalari et al., BMC Bioinformatics 2014, 15:224). Briefly, paired-end reads were aligned by TopHat 2.0.6 against the UCSC hg19 (human) or rn5 (rat) genome (Trapnell et al., Bioinformatics 2009, 25:1105-1111). Gene counts were generated using HTseq software (online at huber.embl.de/users/anders/HTSeq/doc/overview.html) with gene annotation files obtained from Illumina (online at cufflinks.cbcb.umd.edu/igenomes.html). Differential expression between groups was calculated using R package edgeR (Robinson et al., Bioinformatics 2010, 26:139-140). FDR 0.05 and log 2 fold change >=1 or log 2 fold change <=−1 was considered as the cutoff for up and down regulated genes.

Western blotting. Briefly, proteins were separated by 4-15% SDSPAGE, transferred to nitrocellulose membranes (Bio-Rad, Hercules, Calif.), and incubated overnight at 4° C. with antibodies against TGR5 (Santa Cruz Biotechnology), Gas (Abcam, Cambridge, Mass.), and Gαᵢ (Cell Signaling Technology, Danvers, Mass.). Actin antibody was used to assure equal protein loading. Membranes were washed and incubated for 1 hour at room temperature with the corresponding horseradish peroxidase-conjugated (Invitrogen) or IRDYE® 680 or 800 (Odyssey) secondary antibodies. Bands were visualized with the ECL Plus Western Blotting Detection kit (BD Biosciences, San Jose, Calif.) or Odyssey Li-Cor Scanner (Li-Cor, Lincoln, Nebr.).

Immunofluorescence Confocal Microscopy.

Livers from wild type (Harlan Sprague Dawley) and PCK (Mayo colonies) rats; wild type, Pkd2^{WS25/-}, Pkhd1^{del2/del2} and Tgr5^{-/-} mice (all of C57BL/6 background and from Mayo's colonies); healthy human beings and patients with ADPKD and ADPLD (provided by the Mayo Clinical Core and National Disease Research Interchange) were incubated overnight with antibodies to TGR5, acetylated β-tubulin (Sigma), Gαs, and Gαᵢ. Fluorescent secondary antibodies (Molecular Probes, Eugene, Oreg.) were applied for 1 hour at room temperature. Nuclei were stained with Prolong Gold Antifade Reagent with 4-,6-diamidino-2-phenylindole (Invitrogen). A ZeissLSM-510 microscope (Carl Zeiss, Thornwood, N.Y.) was used for analysis.

Immuno-Gold Transmission Electron Microscopy (IG-TEM).

Cholangiocytes were grown to 7-9 days postconfluence on collagen-coated coverslips (BD Biosciences, Sparks, Md.), fixed in 4% paraformaldehyde and 0.2% glutaraldehyde for 1 hour at room temperature, and washed in (i) 0.1 M phosphate buffer (PB; pH 7.2-7.4); (ii) PB containing 0.1% sodium borohydride (10 minutes, 4 times); (iii) PB; (iv) PB containing 0.1% Triton X-100 (5 minutes); and (v) PB (four times). Samples were first incubated for 60 minutes at 4° C. in blocking solution (PBS with 1% FCS) and then overnight at 4° C. with TGR5 (ab72608, Abcam) primary antibodies diluted 1:20 with PBS containing 2% FCS. After six washes with PBS containing 2% FCS, samples were incubated for 1 hour at room temperature with a secondary goat anti-mouse antibody conjugated with ultra-small gold (Electron Microscopy Sciences, 1:100 dilutions). Samples were washed in PBS, post-fixed with 2.5% glutaraldehyde in PB for 2 hours, enhanced with silver enhancement mixture (R-Gent SE-EM) for 30 minutes, and treated with 1% osmium tetroxide for 30 minutes. Samples with omitted primary antibodies served as controls. Samples were dehydrated, embedded in Spurrs resin, and sectioned at 90 nm, and observed using a Joel 12 electron microscope (Joel USA, Peabody, Mass.).

cAMP Production.

Production of cAMP was detected using a Bridge-It cAMP designer cAMP assay (Mediomics, St. Lou is, MO) according to manufacturer's protocol. Cholangiocytes were incubated with TLCA, OA, C1 and C2 (all, 25 μM) for 30 minutes. Doses of TGR5 agonists were chosen based on published data (Masyuk, Am J Physiol. Gastrointestinal and Liver Physiol 2013, 304:G1013-1024; and Keitel et al., Hepatol 2007, 45:695-704). Forskolin (10⁻⁶M for 15 min) served as a control.

Cell Proliferation.

Cell proliferation was determined using the CellTiter 96 AQueous One Solution Cell Proliferation Assay (Promega, Madison, Wis.). Cholangiocytes (n=8 for each cell line) were seeded (2500 cells/well) and grown in regular DMEM/F12 media at 37° C. (5% CO₂ and 100% humidity) for 48 hours. Cells were treated with TLCA, OA, C1, or C2 (all 25 μM) for 24 hours. Epidermal growth factor (EGF, 20 ng/ml) was used as a positive control.

Three-Dimensional Cultures.

Cultured cholangiocytes and cystic bile ducts freshly isolated by microdissection from PCK rats were grown in three-dimensional matrices as described elsewhere (Masyuk et al., Am J Pathol 2014, 184:110-121; and Masyuk et al., Am J Pathol 2004, 165:1719-1730) and treated with TLCA and OA (both, 25 μm) daily. Images were taken at days 1 (i.e., 24 hours after seeding) and 3. The circumferential area of each cyst was measured using ImageJ software (National Institutes of Health, Bethesda, Md.) as described elsewhere (Masyuk et al., Am J Pathol 2014, 184:110-121). Data were expressed as percent change at day 3 compared to day 1.

Treatment Protocol.

PCK rats (n=10) were injected intraperitoneally with OA (25 mg/kg bw) daily. The dose of OA was chosen based on studies described elsewhere (Jeong et al., Biopharm Drug Disposition 2007, 28:51-57). Control PCK rats (n=8) received equal doses of DMSO. Drug concentrations were adjusted to the animal weight weekly. After 6 weeks of treatment, rats were sacrificed and body and organ weights assessed. H&E and picrosirius red collagen stained liver and kidney sections were used to analyze hepatic and renal cysto-fibrotic areas, respectively, as described elsewhere (Masyuk et al., Hepatol 2013, 58:409-421). Hepatic and renal cystic and fibrotic areas were expressed as a percentage of total hepatic or renal parenchyma, respectively.

Development of Double Mutant TGR5^{-/-}:Pkhd1^{del2/del2} Mice.

TGR5^{-/-} mice (Drs. Auwerx and Schoonjans, Lausanne, Switzerland) were crossed with Pkhd1^{del2/del2} mice (Woollard et al., Kidney Int 2007, 72:328-336), and offspring (TGR5^{-/-}:Pkhd1^{del2/±}) were bred to produce TGR5^{-/-}:Pkhd1^{del2/del2} double mutants. Mice were genotyped using a Kappa Mouse Genotyping Kit (Kapabiosystems, Boston, Mass.) with the following primers:

(i)
(TGR5 forward; SEQ ID NO: 1)
GATGGCTGAGAGGCGAAG
(ii)
(TGR5 reverse; SEQ ID NO: 2)
AGAGCCAAGAGGGACAATCC
(iii)
(Pkhd1 forward; SEQ ID NO: 3)
GGACCTTACAATCTTTTTGCCCC
(iv)
(Pkhd1 reverse; SEQ ID NO: 4)
CATCATACAGTTCTCAGACCCCG.

Body weight, liver weight, kidney weight, and cysto-fibrotic areas were analyzed in age-matched 8-month-old littermates of wild type, TGR5^{-/-}, Pkhd1^{del2/del2} and double mutant TGR5^{-/-}:Pkhd1^{del2/del2} mice.

Statistical Analysis.

The data are expressed as the MEAN±SEM. Statistical analysis was performed by Student's t-test, and results were considered statistically significant at p<0.05.

### Example 2—Results

TGR5 is Overexpressed in Cystic Cholangiocytes.

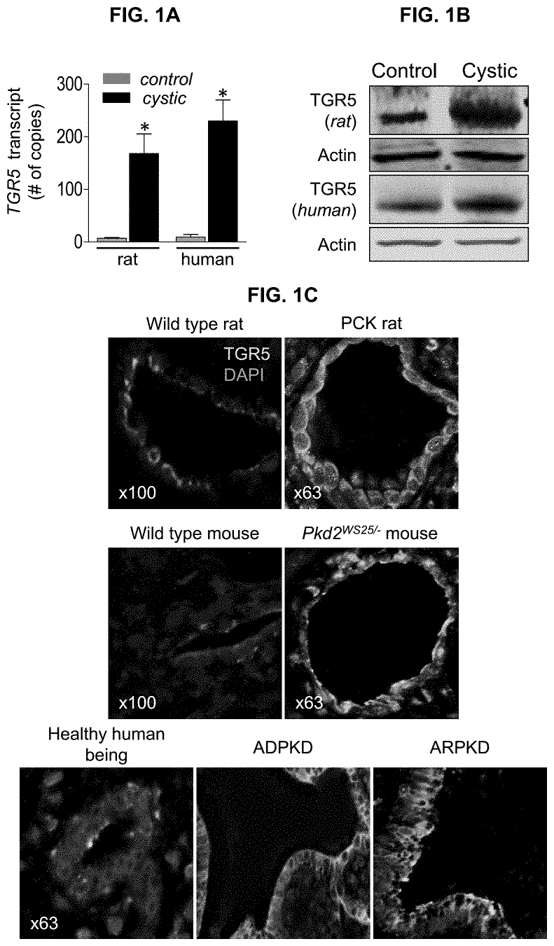

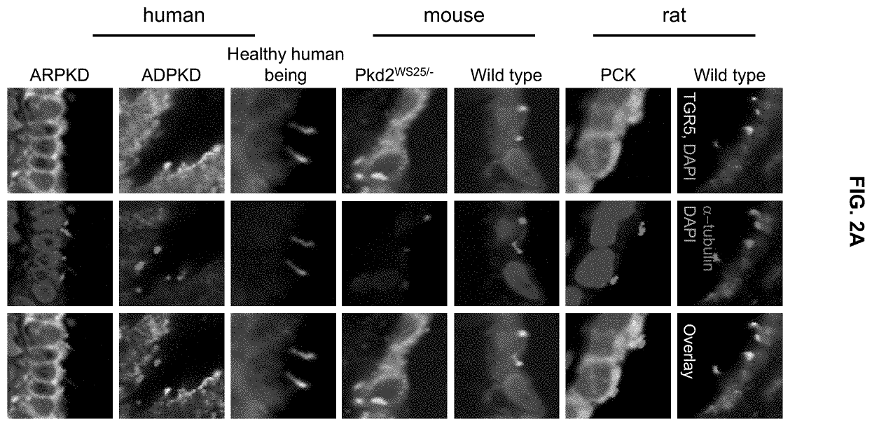

Higher copy numbers of TGR5 transcript and increased levels of TGR5 protein were observed in cystic cholangiocytes as compared to respective controls (FIGS. 1A and 1B). Over-expressed TGR5 also was seen in vivo in cholangiocytes lining liver cysts in animal models of PLD and human patients with ADPKD and ARPKD (FIG. 1C).

TGR5 is Expressed in Cilia of Normal but not Cystic Cholangiocytes.

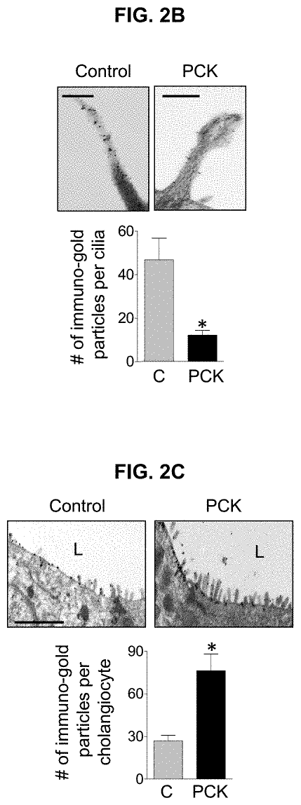

More detailed examination of TGR5 expression revealed that TGR5 is present in primary cilia of control but not cystic cholangiocytes, as detected by confocal and IG-TE microscopy (FIGS. 2A and 2B). TGR5 was markedly overexpressed on the apical membrane of cystic cholangiocytes, however (FIG. 2C).

TGR5 Activation Increases cAMP Levels in Cystic Cholangiocytes.

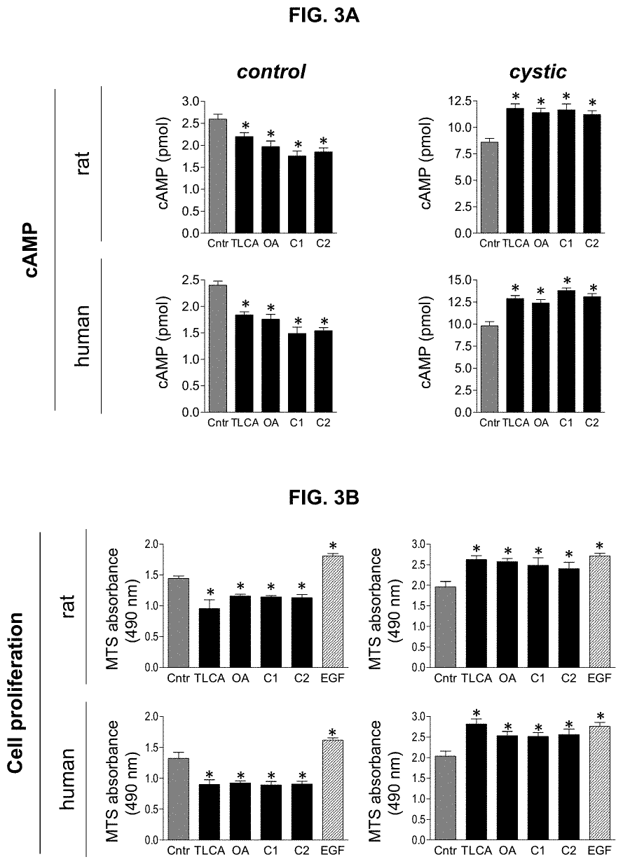

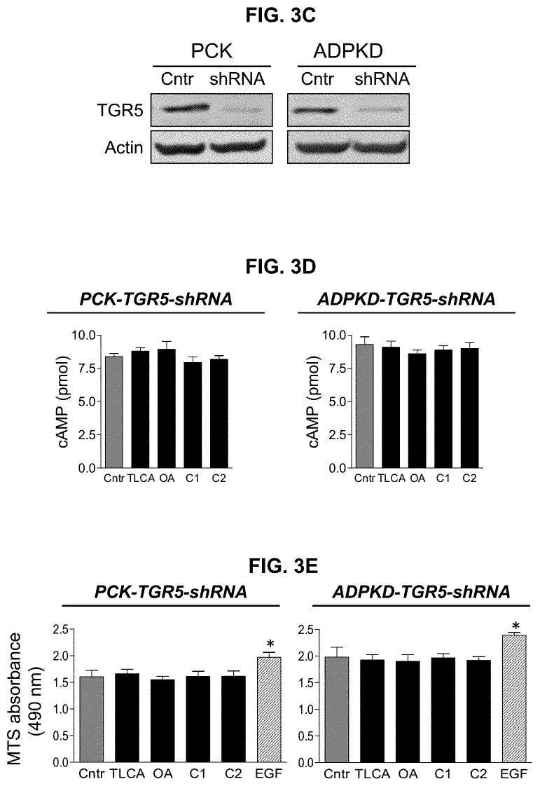

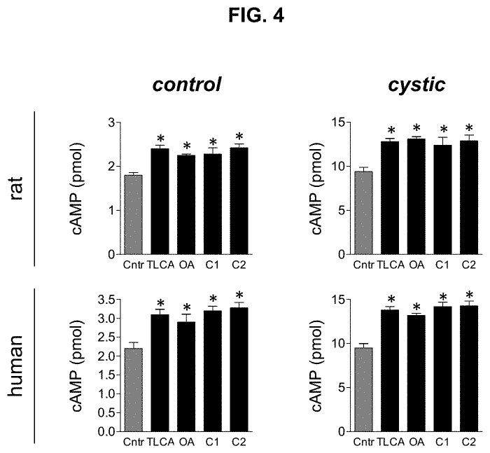

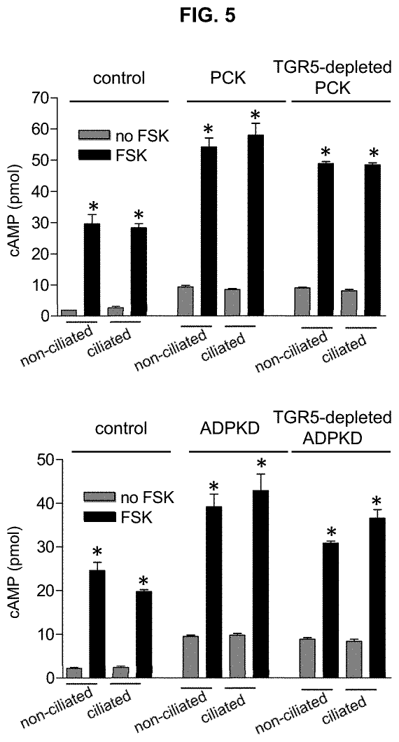

The effects of four TGR5 agonists (TLCA, OA, C1, and C2) on cAMP production was assessed in cholangiocytes grown in culture up to 10 days. By this time, the cholangiocytes developed primary cilia on their apical membranes (Masyuk et al., Am J Pathol 2014, 184:110-121). Agonists of TGR5 decreased cAMP levels in control cholangiocytes (FIG. 3A, left panels) while increasing cAMP production in cystic cholangiocytes (FIG. 3A, right panels). In contrast to ciliated conditions, elevated cAMP was observed in non-ciliated control and cystic cholangiocytes after TGR5 activation (FIG. 4). Forskolin increased cAMP generation independently of the presence or absence of cilia (FIG. 5).

TGR5 Activation Increases Proliferation of Cystic Cholangiocytes.

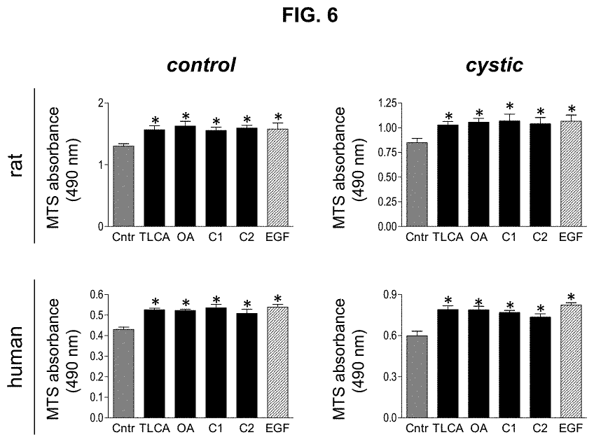

Consistent with findings reported elsewhere (Masyuk et al., Am J Physiol. Gastrointestinal and Liver Physiol 2013, 304:G1013-1024), proliferation of ciliated control cholangiocytes was decreased in response to TGR5 activation (FIG. 3B, left panels). In contrast, TGR5 agonists increased proliferation of cystic cholangiocytes grown under similar conditions (FIG. 3B, right panels). Differential effects of TGR5 agonists on proliferation of control and cystic cholangiocytes also were confirmed using a cell counting approach. Further, non-ciliated cholangiocytes responded to TGR5 activation by increased cell proliferation (FIG. 6).

TGR5 Depletion in Cystic Cholangiocytes Abolished Effects of TGR5 Agonists on cell proliferation and cAMP levels.

To ascertain whether TGR5 is responsible for the observed increase in cAMP production and rate of cell proliferation, TGR5 was depleted in cystic cholangiocytes with specific shRNAs. Western blotting revealed that TGR5 expression was effectively silenced by shRNA (FIG. 3C). Depletion of TGR5 abolished effects of its agonists but not forskolin on cAMP levels and rates of proliferation (FIGS. 3D and 3E; FIG. 6).

TGR5 Activation Accelerates Growth of Cystic Structures In Vitro.

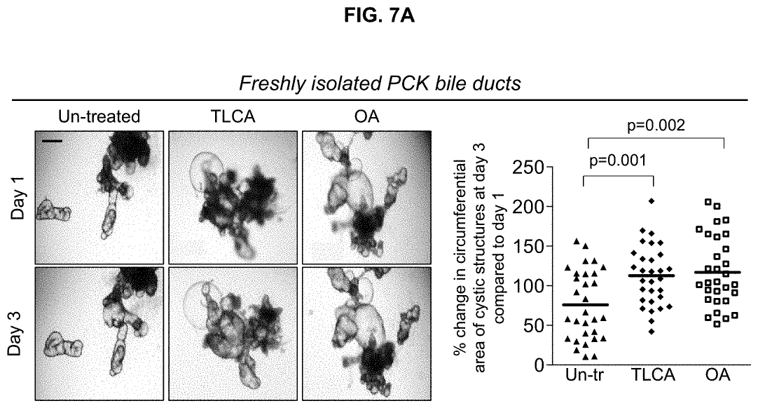

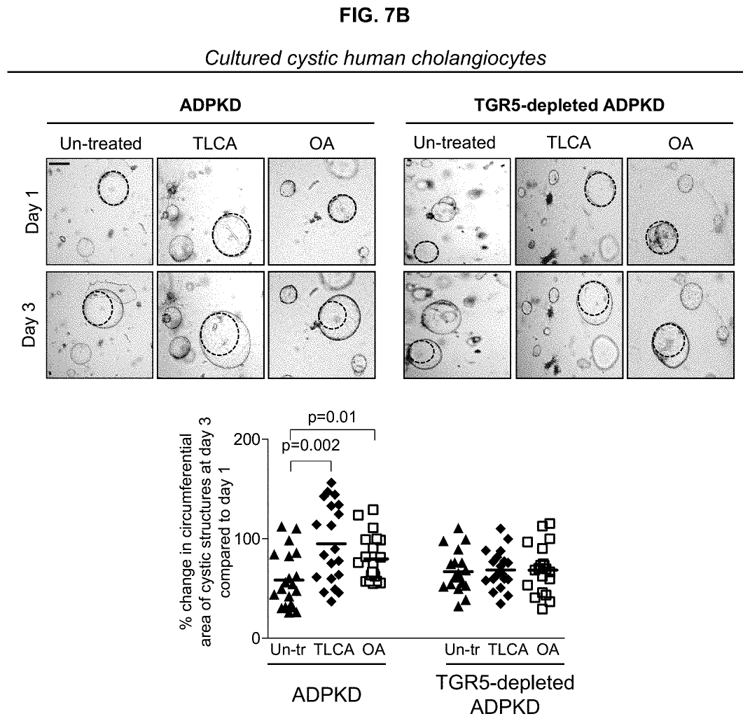

To study the effects of TGR5 agonists on cyst growth, a 3-D model of cystogenesis was employed. Cystic bile ducts from PCK rats expanded progressively over time under basal conditions (FIG. 7A). However, in the presence of TGR5 agonists, accelerated growth of cystic structures was apparent. Similarly, activation of TGR5 enhanced growth of hepatic cystic structures formed by cultured cystic cholangiocytes (FIG. 7B, left panel). Depletion of TGR5 abrogated the effects of TGR5 agonists on cyst growth (FIG. 7B, right panel).

Oleanolic Acid Increases Hepato-Renal Cystogenesis in PLD.

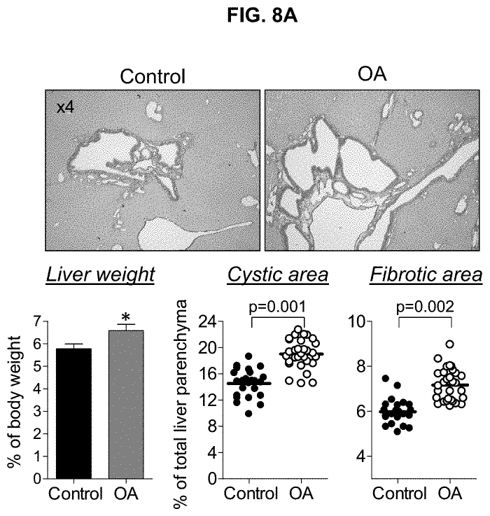

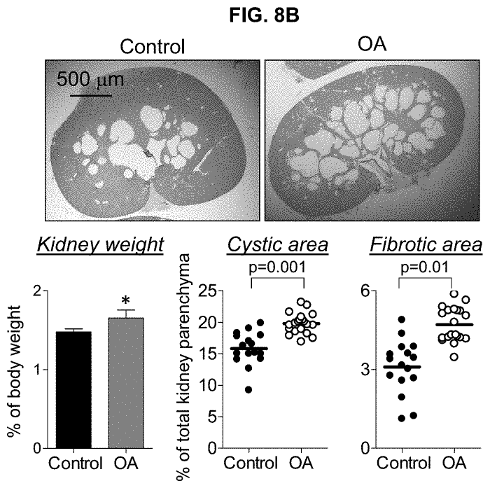

The effects of TGR5 activation on hepato-renal cystogenesis was tested in vivo in PCK rats. OA was well tolerated, and no mortality or toxicity (e.g., hair or weight loss) was observed. Compared to control PCK rats, OA treatment increased: (i) liver weights by 12%; (ii) kidney weights by 11%; (iii) hepatic cystic areas by 31%; (iv) hepatic fibrotic areas by 20%; (v) renal cystic areas by 19%; and (vi) renal fibrotic areas by 30% (FIGS. 8A and 8B).

Genetic Elimination of TGR5 Decreases Hepatic Cystogenesis in PLD.

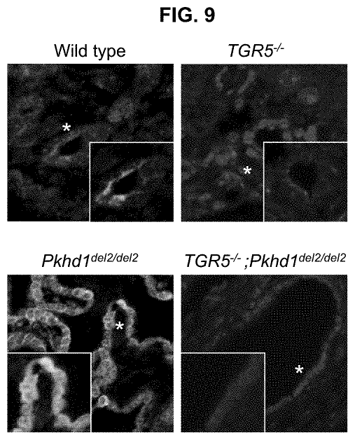

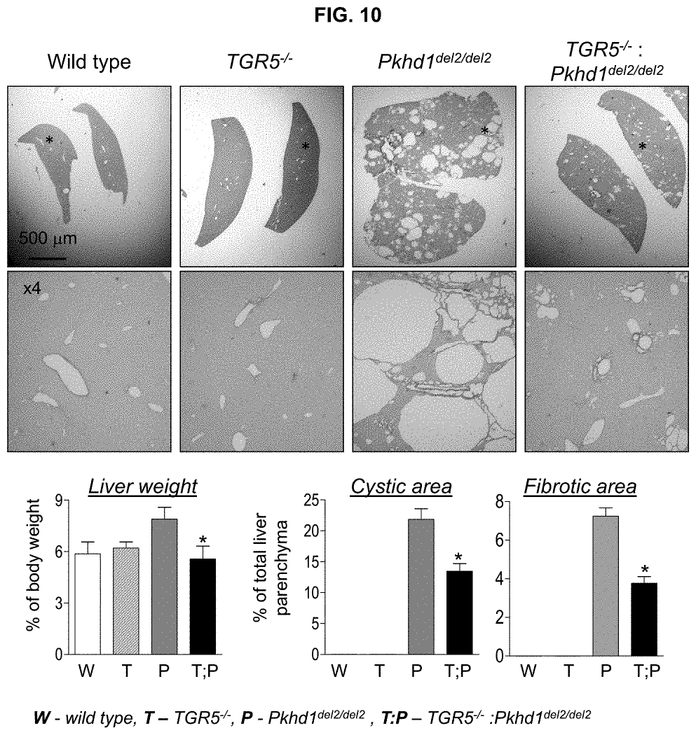

To further examine the involvement of TGR5 in the growth of hepatic cysts, double-mutant TGR5^{-/-}:Pkhd1^{del2/del2} mice were generated. TGR5^{-/-} mice have no morphological abnormalities in the liver, while Pkhd1^{del2/del2} rodents are characterized by the presence of multiple hepatic cysts by 8 months of age (Woollard et al., supra; and Vassileva et al., Biochem J 2006, 398:423-430). Increased TGR5 expression was observed in Pkhd1^{del2/del2} mice as compared to wild type animals, but no TGR5 immunoreactivity was detected in TGR5^{-/-} counterparts and TGR5^{-/-}:Pkhd1^{del2/del2} double mutants (FIG. 9). As compared to Pkhd1^{del2/del2} littermates, TGR5^{-/-}:Pkhd1^{del2/del2} double mutants displayed reductions in: (i) liver weight by 35%; (ii) hepatic cystic areas by 42%; and (iii) hepatic fibrotic areas by 38% (FIG. 10).

TGR5 Activation in Cystic Cholangiocytes Increased Expression of Gαₛ Protein.

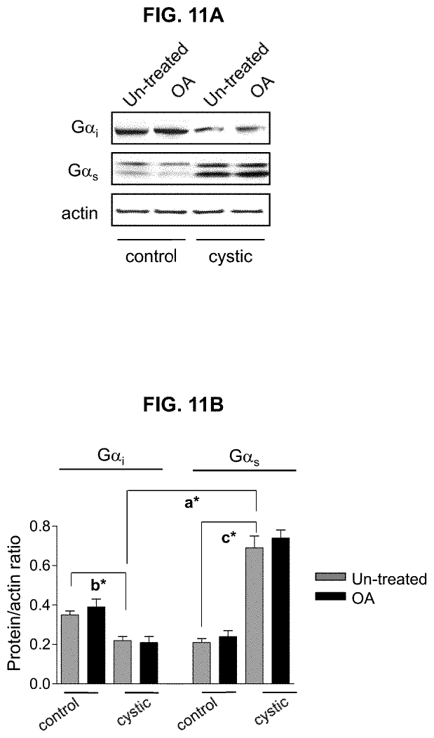

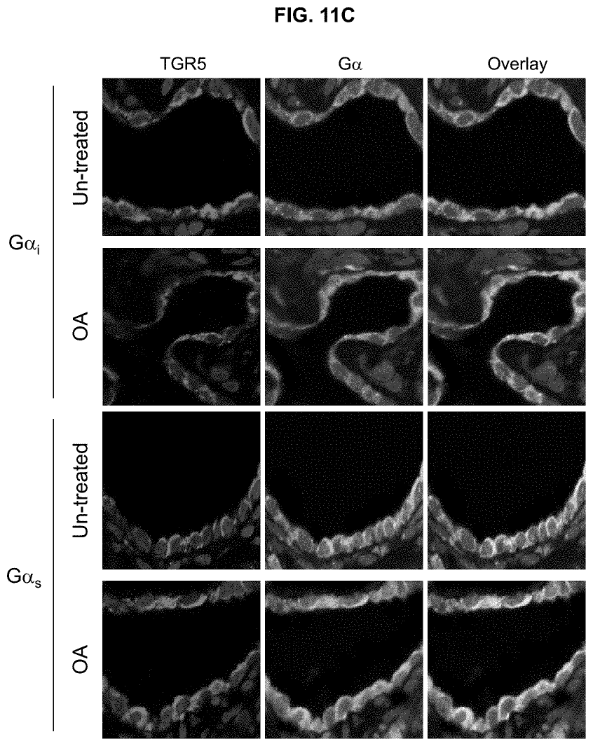

TGR5 is linked to Gαₛ and Gαᵢ proteins (Masyuk et al., Am J Physiol. Gastrointestinal and Liver Physiol 2013, 304:G1013-1024). Thus, the expression of Gαₛ and Gαᵢ proteins was investigated under basal conditions and in response to TGR5 activation by OA. It was observed that: (i) in cystic cholangiocytes, levels of Gαₛ proteins was increased compared to Gαᵢ proteins (FIGS. 11A, 11B [a], and 11C); (ii) expression of Gαᵢ proteins in control cholangiocytes was higher compared to cystic cholangiocytes (FIGS. 11A, 11B[b], and 11C); (iii) expression of Gαₛ proteins in cystic cholangiocytes was increased in comparison with control (FIGS. 11A, 11B[c], and 11C); (iv) OA did not affect the expression of Gαᵢ and Gαₛ proteins in either control or cystic cholangiocytes (FIGS. 11A-11C); and (v) increased coupling of TGR5 and Gαₛ proteins appears to be present in cystic cholangiocytes of PCK rats in response to OA treatment (FIG. 11C).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating polycystic liver disease in a subject, comprising administering to the subject a TGR5 antagonist in an amount effective to reduce at least one symptom of the polycystic liver disease.

2. The method of claim 1, wherein the subject is a human.

3. The method of claim 1, wherein the antagonist is an antibody targeted to TGR5.

4. The method of claim 1, wherein the antagonist is a small molecule.

5. A method for reducing cyst formation in the liver or kidney of a subject, comprising administering to the subject a TGR5 antagonist in an amount effective to reduce the size or number of cysts in the liver or kidney of the subject.

6. The method of claim 5, wherein the subject is diagnosed with PLD.

7. The method of claim 5, wherein the subject is a human.

8. The method of claim 5, wherein the antagonist is an antibody targeted to TGR5.

9. The method of claim 5, wherein the antagonist is a small molecule.

10-17. (canceled)

18. The method of claim 1, wherein the antagonist is an interfering RNA.

19. The method of claim 5, wherein the antagonist is an interfering RNA.


SEQUENCE LISTING (4 sequences)

SEQ ID NO: 1 - DNA, 18 bp, Artificial Sequence
  synthetic oligonucleotide
gatggctgagaggcgaag

SEQ ID NO: 2 - DNA, 20 bp, Artificial Sequence
  synthetic oligonucleotide
agagccaagagggacaatcc

SEQ ID NO: 3 - DNA, 23 bp, Artificial Sequence
  synthetic oligonucleotide
ggaccttacaatctttttgcccc

SEQ ID NO: 4 - DNA, 23 bp, Artificial Sequence
  synthetic oligonucleotide
catcatacagttctcagaccccg